(19) United States
(12) Patent Application Publication
(10) Pub. No.: US 2011/0318299 A1
(43) Pub. Date: Dec. 29, 2011

(54) Composition for Accelerating Nerve Repair

(75) Inventor: Wing-Pong Chan, Taipei City (TW)

(73) Assignee: TAIPEI MEDICAL UNIVERSITY, Taipei City (TW)

(21) Appl. No.: 12/872,833

(22) Filed: Aug. 31, 2010

(30) Foreign Application Priority Data
Jun. 23, 2010 (TW) 099120540

(51) Int. Cl. A61K 38/20; A61K 38/19; A61P 25/00; A61K 35/14

(52) U.S. Cl. 424/85.2; 424/93.72; 424/85.1

(57) ABSTRACT

The present invention discloses a composition for accelerating nerve repair. The composition comprises platelet-rich fibrin, a growth factor and a cytokine. The composition may further comprise a tissue with a stem cell or a neural tube to be a scaffold for promoting nerve generation. The platelet-rich fibrin is obtained from the blood of the mammal to be operated on with a nerve repair.

### FIELD OF THE INVENTION

The present invention relates to a composition, and more particularly, to a composition, for accelerating nerve repair.

### BACKGROUND OF THE INVENTION

A neuron is composed of a cell body, an axon, and a dendrite. The cell body includes basic organelles, such as a cell nuclei, ribosomes, and mitochondria. If the cell body dies, the neuron may also die. The axon is like a cable line, and a chemical electrical signal can be transmitted along the axon and between cells. The axon is covered with myelin, which is like an insulator of the cable line to promote signal transduction. The dendrites are like branches of a tree, for connecting neurons. The neurons can communicate with each other and can communicate external environmental conditions.

The neurons can be classified into types, as sensory neurons, motor neurons, and interneurons, according to their functions. The sensory neurons are used to transmit signals to the central nervous system from the peripheral nervous system, and the motor neurons are used to transmit signals to the peripheral nervous system from the central nervous system. The spinal cord is connected to neurons in the cerebrum by the interneurons.

After neurotmesis, or severe nerve damage, the nerve may be repaired to its original status, but the repair opportunity thereof is very small. Usually, scars form on the repaired nerve, resulting in lower transduction rates. The muscles controlled by neurons may get worse or even lose their functions. Nerve anastomosis is the usual nerve repair operation, and which nerve anastomosis is selected depends on the length of broken nerves. The broken nerves operated on by the nerve anastomosis may result in a long recovery time for patients, with inconvenience to their lifestyles. Artificial neural tubes or donated nerves are also another option. When donated nerves are used as scaffolds, transplant rejection may occur.

### SUMMARY OF THE INVENTION

In view of the aforementioned drawbacks in existing skills or techniques, an object of the present invention is to provide a composition for accelerating nerve repair, so as to shorten the recovery time and generate new nerves without scar formation.

To achieve the above object, the composition for accelerating nerve repair comprises platelet-rich fibrin (PRF), a growth factor and cytokine. The PRF is obtained autologously from the blood of a mammal. The composition further comprises tissue with a stem cell obtained from the same mammal or a neural tube to mix with the composition. The neural tube can comprise an autologous nerve or an artificial neural tube. The tissue with the stem cell or the neural tube can be a scaffold to promote nerve regeneration.

In addition, because the PRF is obtained autologously from the mammal, transplant rejection does not occur. The growth factor may comprise a transforming growth factor (TGF), a platelet-derived growth factor (PDGF), an epidermal growth factor, a vascular endothelial growth factor, or an insulin-like growth factor, or a combination thereof. The cytokine may comprise interleukins or tumor necrosis factors.

The composition for accelerating nerve repair according to the present invention provides one or more of the following advantages:

(1) Because the composition is obtained autologously from the mammal to be surgically operated on, the mammal does not have transplant rejection.

(2) The composition comprises platelet-rich fibrin (PRF). PRF can promote growth factors to activate normal immune responses, accelerate blood vessel regeneration, and at the same time induce aggregation and differentiation of cyclic adult stem cells and mesenchymal stem cells (MSCs) in vivo to speed up tissue regeneration.

### BRIEF DESCRIPTION OF THE DRAWINGS

FIG. 1 is the platelet-rich fibrin obtained autologously from the blood of the dogs after centrifugation at 2500-3500 rpm for 8-12 minutes.

FIG. 2 illustrates the hind leg of each dog is operated on from the ilium lateral angle to the femur 3-10 cm below, with a 7 cm incision to cut the sciatic nerve after anesthetization.

FIG. 3 illustrates the cut sciatic nerve is stitched by an absorbable suture with the autologous PRF, the growth factors, and the cytokines.

FIG. 4 illustrates the sciatic nerve has been recovered and the dogs can walk via using their hind leg one month later.

### DETAILED DESCRIPTION OF THE PREFERRED EMBODIMENTS

The present invention will now be described with some preferred embodiments thereof. It is understood that the experimental data shown in the embodiments are provided only for easy interpretation of the technical means of the invention and should in no means be considered as restriction of the invention.

The composition for accelerating nerve repair of the present invention comprises platelet-rich fibrin (PRF), a growth factor and a cytokine. The PRF may be obtained from blood in the mammal to undergo nerve repair. The composition may further comprise tissue obtained from the same mammal with a stem cell or neural tube. The tissue, with the stem cell and the neural tube, can be mixed with the PRF, the growth factor, and the cytokine, and therefore the tissue with the stem cell or the neural tube can be a scaffold to increase nerve regeneration. The neural tube can be an artificial neural tube or an autologous nerve. If the length of broken nerves is long, the PRF, the growth factor, and the cytokine are mixed with the artificial neural tube to stitch to the site on the broken nerve. When the length of broken nerves is short, the broken nerve can be stitched through end-to-end suture by using the composition mixed with the autologous nerve or only the composition, comprising the PRF, the growth factor, and the cytokine. Preferably, the surface of the stitched nerve can be covered with the composition to further increase nerve repair rates.

The PRF is obtained from blood in a mammal, which is collected in a container holding a separator polyester gel, and then centrifuged at a speed in the range of 1000 to 5000 rpm for 1 to 20 minutes. The centrifuge time is adjusted according to the centrifuge speed. Additionally, the blood may be centrifuged twice so as to obtain different concentrations of PRF.

The growth factor may comprise a transforming growth factor (TGF), a platelet-derived growth factor (PDGF), an epidermal growth factor, a vascular endothelial growth factor, or an insulin-like growth factor, or a combination thereof. The transforming growth factor, such as TGFβ-1, can promote cell growth and differentiation and regulate immune functions. The cytokine may comprise interleukins, such as IL-6, IL-β or IL-4, or tumor necrosis factors, such as TNF-α. Therefore, the composition according to the present invention has the cytokines needed for inflammatory responses and repair responses to promote normal immune responses in mammals. Additionally, parts of the cytokines can protect neurons and glial cells from apoptosis. For example, apoptosis can be prevented by decreasing free radical formation. The trophic factors in the PRF of the present invention not only can promote development of nerve axons, but can regulate neurotransmitter secretion and synaptic plasticity.

In the preferred embodiment, dogs are selected for use as laboratory animals. The dogs are divided into two groups, the control group and the experimental group (3 dogs in each group). Each dog has operations on the sciatic nerve of the hind leg. The composition of the present invention is not implanted into the dogs in the control group, but is implanted into the dogs in the experimental group. The experimental period is six months.

The PRF of the composition of the present invention is obtained from autologous blood of each dog. Six ml of blood is drawn from each of the dogs. The blood is collected in a container, such as a centrifuge tube, holding a separator polyester gel, and in the present embodiment, the centrifuge tube is used for containing the blood. The centrifuge tube containing the blood is centrifuged at a speed in the range of 1000 to 5000 rpm for 1 to 20 minutes, and preferably, also at 2500 to 3500 rpm for 8 to 12 minutes. The centrifuge time may be adjusted according to the centrifuge speed. After centrifugation, jelly-like PRF is obtained in the middle part of the centrifuge tube, and then sterile forceps are used to clip the jelly-like PRF out. All steps in the preparation of the PRF are performed under standard disinfection procedures. The 6 ml of whole blood may yield 1-1.5 ml of PRF. The PRF has growth factors and cytokines required by the animal, and specific growth factors and cytokines can also be additionally added to the obtained PRF, as shown in FIG. 1.

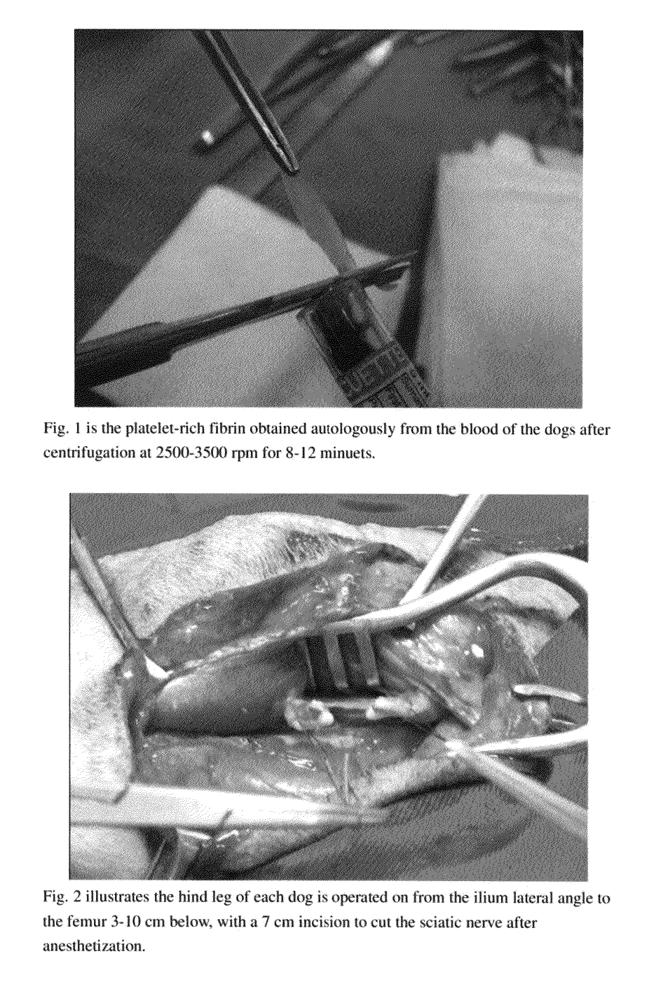

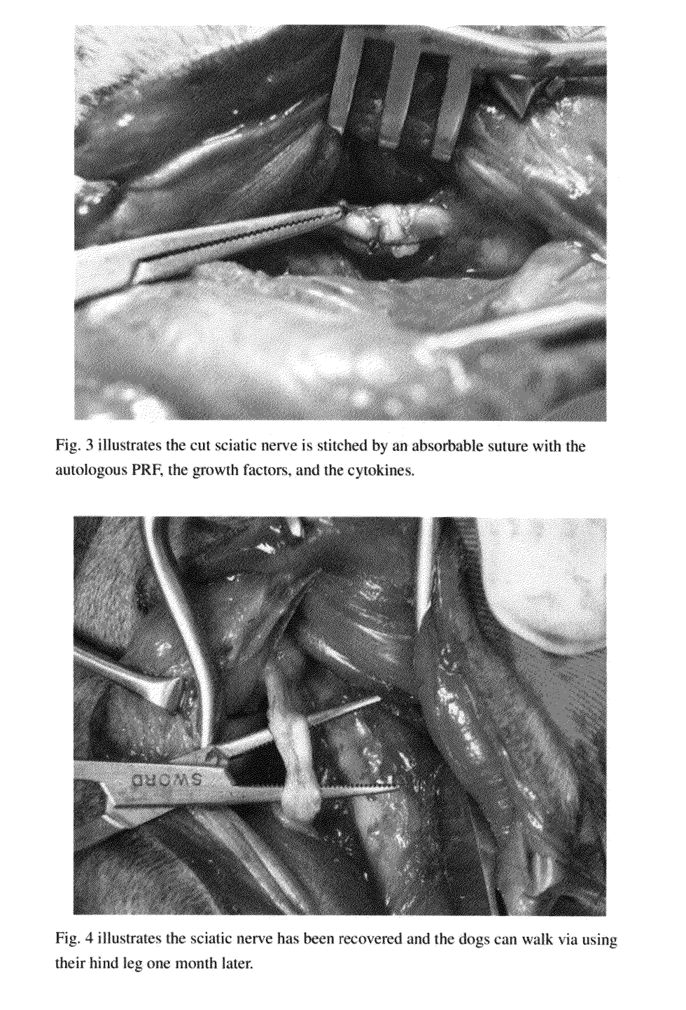

In the present embodiment, each dog has operations on the sciatic nerve. After the dogs are anesthetized, the hind leg of each dog is operated on from the ilium lateral angle to the femur 3-10 cm below, with a 7 cm incision, as shown in FIG. 2. The sciatic nerve is shown after dissection of various muscles bluntly and sequentially. Further, the sciatic nerve is cut off and then stitched by an absorbable suture with the autologous PRF, the growth factors, and the cytokines, as shown in FIG. 3. Each layer of the muscles is stitched sequentially by continuous suture.

The results show that about one month later, the dogs in the experimental group can walk via using their hind leg, which indicates that the sciatic nerve thereof has recovered, as shown in FIG. 4. In the dogs in the control group, it takes more than three months for the cut sciatic nerve to be repaired. As a result, the repair time in the dogs with the composition of the present invention is faster than that without the composition.

The present invention has been described with some preferred embodiments thereof, and it is understood that many changes and modifications in the described embodiments can be carried out without departing from the scope and the spirit of the invention, which is intended to be limited only by the appended claims.

What is claimed is:

1. A composition for accelerating nerve repair, comprising:
platelet-rich fibrin (PRF);
a growth factor; and
a cytokine.

2. The composition for accelerating nerve repair as claimed in claim 1, further comprising tissue with a stem cell or a neural tube.

3. The composition for accelerating nerve repair as claimed in claim 1, wherein the growth factor comprises a transforming growth factor, a platelet-derived growth factor, an epidermal growth factor, a vascular endothelial growth factor, or an insulin-like growth factor, or a combination thereof.

4. The composition for accelerating nerve repair as claimed in claim 1, wherein the cytokine comprises an interleukin or a tumor necrosis factor.

5. The composition for accelerating nerve repair as claimed in claim 1, wherein the PRF is obtained from blood after centrifugation.

6. The composition for accelerating nerve repair as claimed in claim 5, wherein the blood is collected in a container containing a separator polyester gel.

7. The composition for accelerating nerve repair as claimed in claim 6, wherein a rotational speed of the centrifugation ranges between 1000 and 1500 rpm.

8. The composition for accelerating nerve repair as claimed in claim 7, wherein rotational time of the centrifugation ranges between 1 and 20 minutes.
